(19) United States
(12) Patent Application Publication
(10) Pub. No.: US 2007/0293508 A1
(43) Pub. Date: Dec. 20, 2007

(54) Crf Receptor Antagonists and Methods Relating Thereto

(75) Inventors: John Williams, San Diego, CA (US); Zhiyong Luo, New York, NY (US); John Edward Tellew, La Jolla, CA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignees: SB Pharmco Puerto Rico Inc and Neurocrine Biosciences, Inc., San Diego, CA (US); Neurocrine Biosciences, Inc.,, San Diego, CA (US); SB Pharmco Puerto Rico, Hato Rey, PR (US)

(21) Appl. No.: 10/596,651

(22) PCT Filed: Dec. 20, 2004
(86) PCT No.: PCT/IB04/04242

§ 371 Date: May 29, 2007

Related U.S. Application Data
(60) Provisional application No. 60/532,032, filed on Dec. 22, 2003.

(51) Int. Cl. A61K 31/4965; A61P 25/00; C07D 471/00

(52) U.S. Cl. 514/255.05; 544/346

(57) ABSTRACT

CRF receptor antagonists are disclosed which have utility in the treatment of a variety of disorders in mammals, including the treatment of disorders, such as stroke, manifesting hypersecretion of CRF. The CRF receptor antagonists of this invention have the following structure: 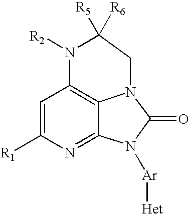
including stereoisomers, prodrugs and pharmaceutically acceptable salts thereof, wherein R₁, R₂, R₅, Ar, and Het are as defined herein. Compositions containing a CRF receptor antagonist in combination with a pharmaceutically acceptable carrier are also disclosed, as well as methods for use of the same.

### CROSS-REFERENCE TO RELATED APPLICATION

This application claims priority to U.S. Provisional Application Ser. No. 60/532,032, filed Dec. 22, 2003, the entire disclosure of which is incorporated by reference herein.

### TECHNICAL FIELD

This invention relates generally to CRF receptor antagonists and to methods of treating disorders by administration of such antagonists to a mammal in need thereof.

### BACKGROUND OF THE INVENTION

The first corticotropin-releasing factor (CRF) was isolated from ovine hypothalami and identified as a 41-amino acid peptide (Vale et al., Science 213:1394-1397, 1981). Subsequently, sequences of human and rat CRF were isolated and determined to be identical, but different from ovine CRF in 7 of the 41 amino acid residues (Rivier et al., Proc. Natl. Acad. Sci. USA 80:4851, 1983; Shibahara et al., EMBO J. 2:775, 1983).

CRF has been found to produce profound alterations in endocrine, nervous and immune system function. CRF is believed to be the major physiological regulator of the basal and stress-release of adrenocorticotropic hormone (“ACTH”), β-endorphin, and other pro-opiomelanocortin (“POMC”)-derived peptides from the anterior pituitary (Vale et al., Science 213:1394-1397, 1981). Briefly, CRF is believed to initiate its biological effects by binding to a plasma membrane receptor which has been found to be distributed throughout the brain (DeSouza et al., Science 224:1449-1451, 1984), pituitary (DeSouza et al., Methods Enzymol. 124:560, 1986; Wynn et al., Biochem. Biophys. Res. Comm. 110:602-608, 1983), adrenals (Udelsman et al., Nature 319:147-150, 1986) and spleen (Webster, E. L., and E. B. DeSouza, Endocrinology 122:609-617, 1988). The CRF receptor is coupled to a GTP-binding protein (Perrin et al., Endocrinology 118:1171-1179, 1986) which mediates CRF-stimulated increase in intracellular production of cAMP (Bilezikjian, L. M., and W. W. Vale, Endocrinology 113:657-662, 1983). The receptor for CRF has now been cloned from rat (Perrin et al., Endo 133(6):3058-3061, 1993), and human brain (Chen et al., PNAS 90(19):8967-8971, 1993; Vita et al., FEBS 335(1):1-5, 1993). This receptor is a 415 amino acid protein comprising seven membrane spanning domains. A comparison of identity between rat and human sequences shows a high degree of homology (97%) at the amino acid level.

In addition to its role in stimulating the production of ACTH and POMC, CRF is also believed to coordinate many of the endocrine, autonomic, and behavioral responses to stress, and may be involved in the pathophysiology of affective disorders. Moreover, CRF is believed to be a key intermediary in communication between the immune, central nervous, endocrine and cardiovascular systems (Crofford et al., J. Clin. Invest. 90:2555-2564, 1992; Sapolsky et al., Science 238:522-524, 1987; Tilders et al., Regul. Peptides 5:77-84, 1982). Overall, CRF appears to be one of the pivotal central nervous system neurotransmitters and plays a crucial role in integrating the body's overall response to stress.

Administration of CRF directly to the brain elicits behavioral, physiological, and endocrine responses identical to those observed for an animal exposed to a stressful environment. For example, intracerebroventricular injection of CRF results in behavioral activation (Sutton et al., Nature 297:331, 1982), persistent activation of the electroencephalogram (Ehlers et al., Brain Res. 278:332, 1983), stimulation of the sympathoadrenomedullary pathway (Brown et al., Endocrinology 110:928, 1982), an increase of heart rate and blood pressure (Fisher et al., Endocrinology 110:2222, 1982), an increase in oxygen consumption (Brown et al., Life Sciences 30:207, 1982), alteration of gastrointestinal activity (Williams et al., Am. J. Physiol. 253:G582, 1987), suppression of food consumption (Levine et al., Neuropharmacology 22:337, 1983), modification of sexual behavior (Sirinathsinghji et al., Nature 305:232, 1983), and immune function compromise (Irwin et al., Am. J. Physiol. 255:R744, 1988). Furthermore, clinical data suggests that CRF may be hypersecreted in the brain in depression, anxiety-related disorders, and anorexia nervosa. (DeSouza, Ann. Reports in Med. Chem. 25:215-223, 1990). Accordingly, clinical data suggests that CRF receptor antagonists may represent novel antidepressant and/or anxiolytic drugs that may be useful in the treatment of the neuropsychiatric disorders manifesting hypersecretion of CRF.

The first CRF receptor antagonists were peptides (see, e.g., Rivier et al., U.S. Pat. No. 4,605,642; Rivier et al., Science 224:889, 1984). While these peptides established that CRF receptor antagonists can attenuate the pharmacological responses to CRF, peptide CRF receptor antagonists suffer from the usual drawbacks of peptide therapeutics including lack of stability and limited oral activity. Some published patent documents include US2002143008, U.S. Pat. No. 6,348,466, WO2001083486, and WO2000027850, all of which disclose tetraazaacenaphthylene compounds as CRF antagonists.

Due to the physiological significance of CRF, the development of biologically-active small molecules having significant CRF receptor binding activity and which are capable of antagonizing the CRF receptor remains a desirable goal. Such CRF receptor antagonists would be useful in the treatment of endocrine, psychiatric and neurological conditions or illnesses, including stress-related disorders in general.

While significant strides have been made toward achieving CRF regulation through administration of CRF receptor antagonists, there remains a need in the art for effective small molecule CRF receptor antagonists. There is also a need for pharmaceutical compositions containing such CRF receptor antagonists, as well as methods relating to the use thereof to treat, for example, stress-related disorders. The present invention fulfills these needs, and provides other related advantages.

### SUMMARY OF THE INVENTION

In brief, this invention is generally directed to CRF receptor antagonists, and more specifically to CRF receptor antagonists having the following general structure (I): 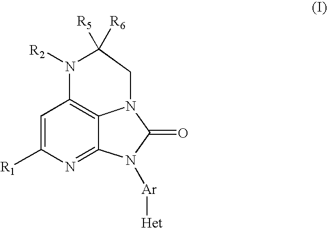
including stereoisomers, prodrugs and pharmaceutically acceptable salts thereof, wherein R₁, R₂, R₅, Ar, and Het are as defined below.

The CRF receptor antagonists of this invention may have utility over a wide range of therapeutic applications, and may be used to treat a variety of disorders or illnesses, including stress-related disorders. Such methods include administering an effective amount of a CRF receptor antagonist of this invention, preferably in the form of a pharmaceutical composition, to an animal in need thereof. Accordingly, in another embodiment, pharmaceutical compositions are disclosed containing one or more CRF receptor antagonists of this invention in combination with a pharmaceutically acceptable carrier and/or diluent.

These and other aspects of the invention will be apparent upon reference to the following detailed description. To this end, various references are set forth herein which describe in more detail certain procedures, compounds and/or compositions, and are hereby incorporated by reference in their entirety.

### DETAILED DESCRIPTION OF THE INVENTION

The present invention is directed generally to compounds useful as corticotropin-releasing factor (CRF) receptor antagonists.
In a first embodiment, the CRF receptor antagonists of this invention have the following structure (I): 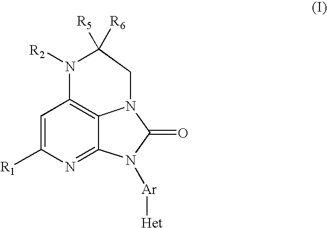
including pharmaceutically acceptable salts, esters, solvates, stereoisomers and prodrugs thereof,
wherein:

R₁ and R₂ are the same or different and independently are hydrogen, alkyl, or substituted alkyl;

Ar is phenyl, phenyl substituted by 1 or 2 R₃, pyridyl or pyridyl substituted by 1 or 2 R₃;

R₃ at each occurrence is independently alkyl, substituted alkyl, alkoxy, substituted alkoxy, cyano, halogen, alkylsulfinyl, or alkylsulfonyl;

Het is heterocycle or heterocycle substituted with 1 or 2 R₄;

R₄ at each occurrence is independently alkyl, substituted alkyl, alkoxy, substituted alkoxy, halogen, cyano or oxo; and

R₅ and R₆ are the same or different and independently are hydrogen, alkyl or substituted alkyl.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclopropyl, —CH₂-cyclobutyl, —CH₂-cyclopentyl, —CH₂-cyclohexyl, and the like; while unsaturated cyclic alkyls include cyclopentenyl and cyclohexenyl, and the like. Cyclic alkyls, also referred to as “homocyclic rings,” and include di- and poly-homocyclic rings such as decalin and adamantyl. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like.

“Alkylidenyl” represents a divalent alkyl from which two hydrogen atoms are taken from the same carbon atom, such as ═CH₂, ═CHCH₃, ═CHCH₂CH₃, ═C(CH₃)CH₂CH₃, and the like.

“Aryl” means an aromatic carbocyclic moiety such as phenyl or naphthyl.

“Arylalkyl” means an alkyl having at least one alkyl hydrogen atoms replaced with an aryl moiety, such as benzyl (i.e., —CH₂phenyl), —CH₂-(1 or 2-naphthyl), —(CH₂)₂phenyl, —(CH₂)₃phenyl, —CH(phenyl)₂, and the like.

“Heteroaryl” means an aromatic heterocycle ring of 5- to 10-members and having at least one heteroatom selected from nitrogen, oxygen and sulfur, and containing at least 1 carbon atom, including both mono- and bicyclic ring systems. Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocycle ring”) means a 5- to 7-membered monocyclic, or 7- to 14-membered polycyclic, heterocycle ring which is either saturated, unsaturated or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring as well as tricyclic (and higher) heterocyclic rings. The heterocycle may be attached via any heteroatom or carbon atom. Heterocycles include heteroaryls as defined above. Thus, in addition to the aromatic heteroaryls listed above, heterocycles also include (but are not limited to) morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

“Oxo” means an oxygen double bonded to a carbon (═O).

The term “substituted” as used herein means any of the above groups (i.e., alkyl, aryl, arylalkyl, heteroaryl, heteroarylalkyl, heterocycle or heterocyclealkyl) wherein at least one hydrogen atom is replaced with a substituent. In the case of a keto substituent (“—C(═O)—”) two hydrogen atoms are replaced. “Substituents” within the context of this invention include halogen, hydroxy, cyano, nitro, amino, alkylamino, dialkylamino, alkyl, alkoxy, thioalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b} —NRₐSO₂R_{b}, —ORₐ, —C(═O)Rₐ —C(═O)ORₐ, —C(═O)NRₐR_{b}, —OC(═O)NRₐR_{b}, —SH, —SRₐ, —SORₐ, —S(═O)₂Rₐ, —OS(═O)₂Rₐ, —S(═O)₂ORₐ, wherein Rₐ and R_{b} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

“Halogen” means fluoro, chloro, bromo and iodo.

“Haloalkyl” means an alkyl having at least one hydrogen atom replaced with halogen, such as trifluoromethyl and the like. Haloalkyl is a specific embodiment of substituted alkyl, wherein alkyl is substituted with one or more halogen atoms.

“Alkoxy” means an alkyl moiety attached through an oxygen bridge (i.e., —O-alkyl) such as —O-methyl, —O-ethyl, and the like.

“Thioalkyl” means an alkyl moiety attached through a sulfur bridge (i.e., —S-alkyl) such as —S-methyl, —S-ethyl, and the like.

“Alkylamino” and “dialkylamino” mean one or two alkyl moieties attached through a nitrogen bridge (i.e., —NHalkyl or —N(alkyl)(alkyl)) such as methylamino, ethylamino, dimethylamino, diethylamino, and the like.

“Hydroxyalkyl” means an alkyl substituted with at least one hydroxyl group.

“Alkylsulfonyl or alkylsulfinyl” represents an alkyl substituted with a —S(═O)₂— or —S(═O)— functionality, respectively.

Embodiments of this invention presented herein are for purposes of example and not for purposes of limitation. In a first embodiment of the invention, Ar is phenyl optionally substituted by R₃ n times where n is 0, 1, or 2 in the following structure (II), and in a further embodiment Ar is pyridyl optionally substituted by R₃ n times where n is 0, 1, or 2 in the following structure (III):

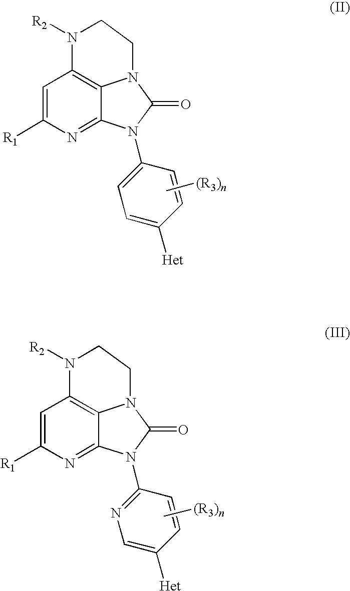

In another embodiment, compounds of this invention have the following structure (IV) when Ar is phenyl and Het is pyridyl optionally substituted with R₄ m times where m is 0, 1 or 2.

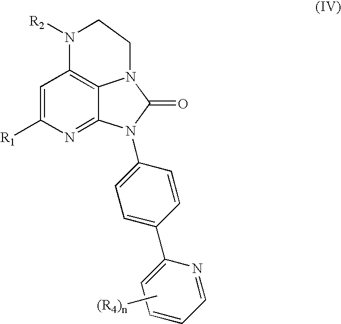

The compounds of the present invention may generally be utilized as the free base. Alternatively, the compounds of this invention may be used in the form of acid addition salts. Acid addition salts of the free base amino compounds of the present invention may be prepared by methods well known in the art, and may be formed from organic and inorganic acids. Suitable organic acids include maleic, fumaric, benzoic, ascorbic, succinic, methanesulfonic, acetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Thus, the term “pharmaceutically acceptable salt” of structure (I) is intended to encompass any and all acceptable salt forms.

In general, the compounds of structure (I) may be made according to the organic synthesis techniques known to those skilled in this field, as well as by the representative methods set forth in the Examples. For example, the synthesis of structure (I) may generally proceed according to the following Reaction Schemes 1 and 2.

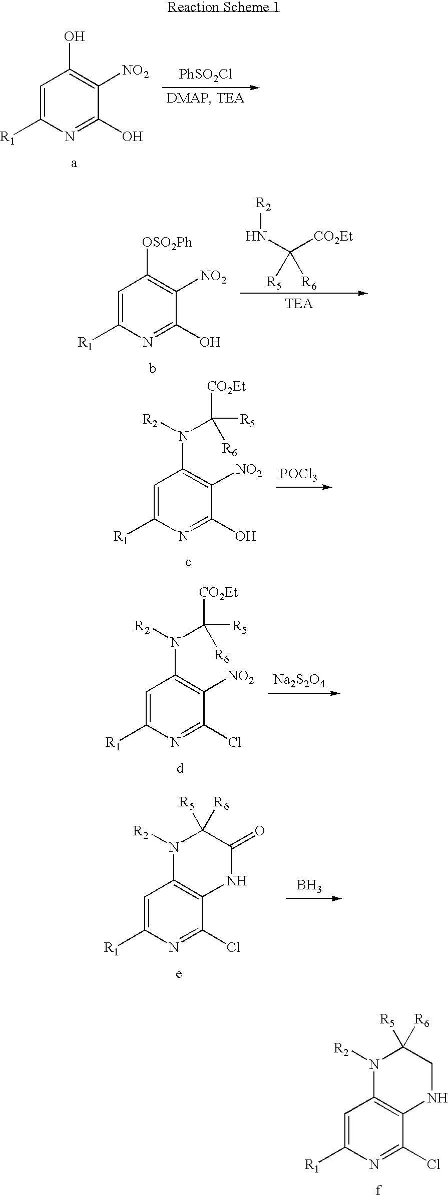

Pyridine a in the presence of DMAP and TEA reacts with benzenesulfonyl chloride to form pyridylphenylsulfonate b. Condensation with alkyl amino acid ester in the presence of base gives aminopyridine c. The 2-chloro derivative d is obtained after reaction of c with phosphorus oxychloride and undergoes ring closure to form the tetrahydropyridopyrazine e after reaction with sodium hydrosulfite. Compound f is obtained after reduction with, for example, borane.

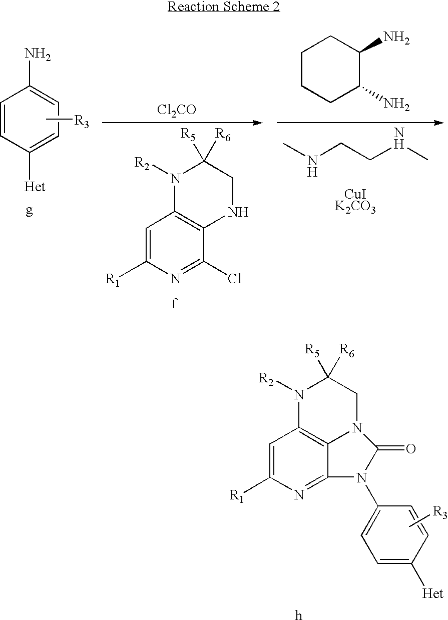

To a solution of aniline g and DIEA in dry DCM is added phosgene. Reaction proceeds overnight, and after evaporation of solvent, tetrahydropyridopyrazine f (Reaction Scheme 1) and DIEA are added in dry DCM. The resulting mixture is stirred and quenched at completion with water. The residue of the dried organic layer is dissolved in 1,4-dioxane, mixed with Cul, K₂CO₃, trans-1,2-diaminocyclohexane and N,N′-dimethylethylenediamine, and allowed to react overnight in a sealed tube at elevated temperature to obtain compound h after purification. In certain embodiments of the invention, the benzene ring of Cpd g can be replaced by pyridine to afford the N-linked pyridyl analog of Cpd h.

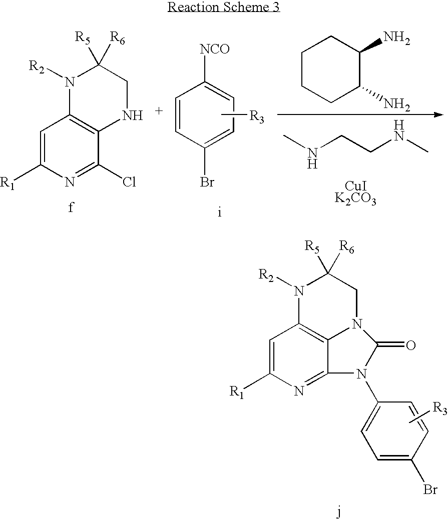

Addition of the phenyl ring to the pyrazolopyridine can be achieved starting with the isocyanate. In Reaction Scheme 3, the (optionally) substituted 4-bromophenylisocyanate i reacts with compound f (Reaction Scheme 1) prior to addition of the N-arylation reagents Cul, K₂CO₃ and the diamines. After purification, compound j is obtained.

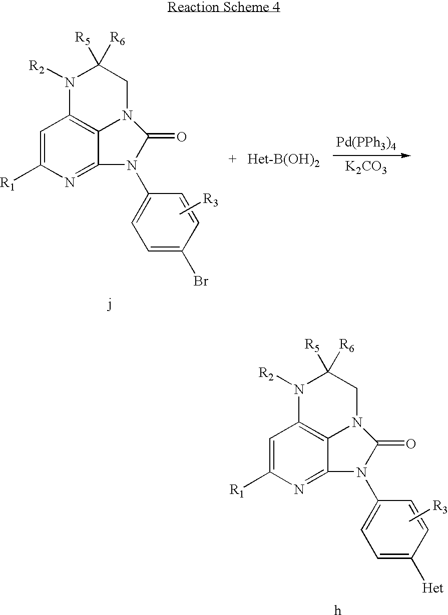

A mixture of compound j with heteroaryl boronic acid, palladium tetrakis(trisphenylphosphine) and K₂CO₃ will react with time at elevated temperature to yield compound h.

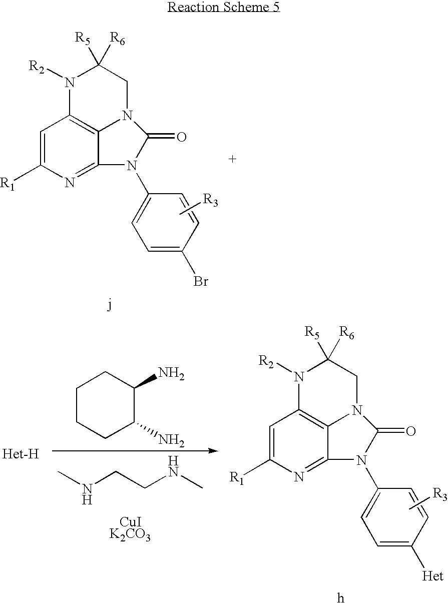

The general procedure of Cul-mediated coupling can be employed in the direct reaction of compound j with a heteroarene, Cul, diamines and K₂CO₃ to obtain compound h. In this procedure, the heteroarene must bear an NH group, the nitrogen atom of which becomes coupled to the aryl group of the final product h.

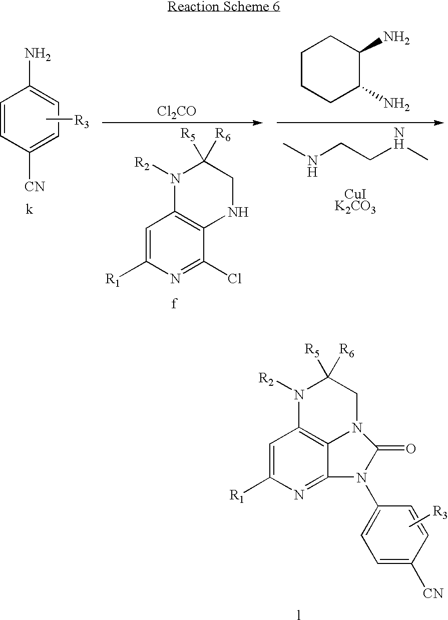

Synthesis of the distal 4-cyanophenyl compound l gives a versatile compound from the which the invention can be realized via further reaction at the cyano functionality. In Reaction Scheme 6, the 4-cyanoaniline k is mixed with phosgene in the presence of base prior to addition of pyrazolopyridine f and the N-arylation reactants, Cul, K₂CO₃, and the diamines from which compound l is obtained.

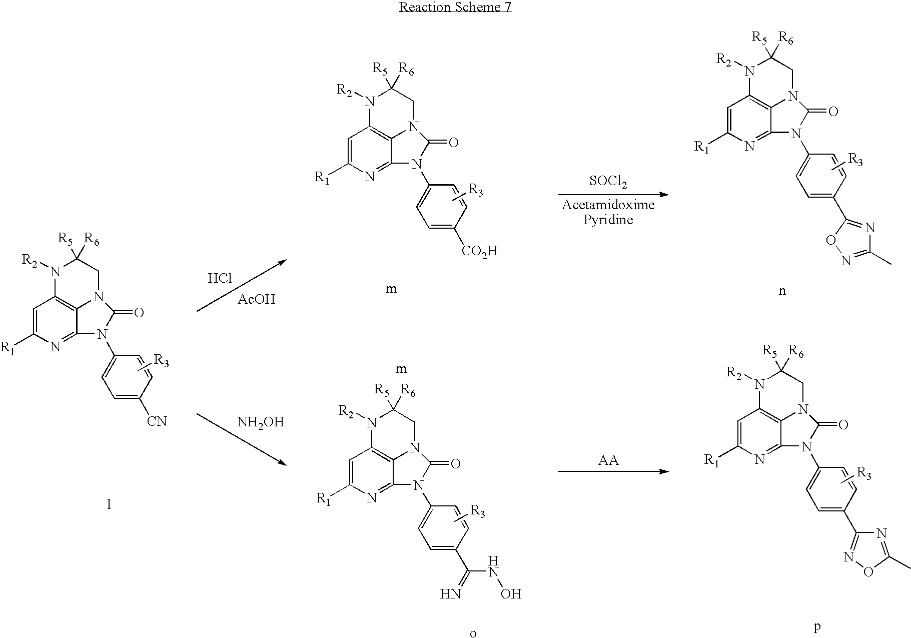

From the 4-cyanophenyl compound l, synthesis of the oxadiazoles n and p can proceed through the acid (m) or the hydroxylamine adduct (o.) In the former case, reaction of compound l with acid forms the carboxylic acid which reacts with thionyl chloride, acetamidoxime, and pyridine to form the 5-methyl-1,2,4-oxadiazole-3-yl adduct n. Alternatively, reaction of compound l with hydroxylamine and further reaction with acetic anhydride (AA) gives the 3-methyl-1,2,4-oxadiazole-5-yl adduct p.

The effectiveness of a compound as a CRF receptor antagonist may be determined by various assay methods. Suitable CRF antagonists of this invention are capable of inhibiting the specific binding of CRF to its receptor and antagonizing activities associated with CRF. A compound of structure (I) may be assessed for activity as a CRF antagonist by one or more generally accepted assays for this purpose, including (but not limited to) the assays disclosed by DeSouza et al. (J. Neuroscience 7:88, 1987) and Battaglia et al. (Synapse 1:572, 1987). As mentioned above, suitable CRF antagonists include compounds which demonstrate CRF receptor affinity. CRF receptor affinity may be determined by binding studies that measure the ability of a compound to inhibit the binding of a radiolabeled CRF (e.g., [¹²⁵I]tyrosine-CFR) to its receptor (e.g., receptors prepared from rat cerebral cortex membranes). The radioligand binding assay described by DeSouza et al. (supra, 1987) provides an assay for determining a compound's affinity for the CRF receptor. Such activity is typically calculated from the IC₅₀ as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22:3099, 1973).

In addition to inhibiting CRF receptor binding, a compound's CRF receptor antagonist activity may be established by the ability of the compound to antagonize an activity associated with CRF. For example, CRF is known to stimulate various biochemical processes, including adenylate cyclase activity. Therefore, compounds may be evaluated as CRF antagonists by their ability to antagonize CRF-stimulated adenylate cyclase activity by, for example, measuring cAMP levels. The CRF-stimulated adenylate cyclase activity assay described by Battaglia et al. (supra, 1987) provides an assay for determining a compound's ability to antagonize CRF activity. Accordingly, CRF receptor antagonist activity may be determined by assay techniques which generally include an initial binding assay (such as disclosed by DeSouza (supra, 1987)) followed by a cAMP screening protocol (such as disclosed by Battaglia (supra, 1987)).

With reference to CRF receptor binding affinities, CRF receptor antagonists of this invention have a Kᵢ of less than 10 μM. In a preferred embodiment of this invention, a CRF receptor antagonist has a Kᵢ of less than 1 μM, and more preferably less than 0.25 μM (i.e., 250 nM). As set forth in greater detail below, the Kᵢ values may be assayed by the methods set forth in Example 7.

CRF receptor antagonists of the present invention may demonstrate activity at the CRF receptor site, and may be used as therapeutic agents for the treatment of a wide range of disorders or illnesses including endocrine, psychiatric, and neurological disorders or illnesses. More specifically, CRF receptor antagonists of the present invention may be useful in treating physiological conditions or disorders arising from the hypersecretion of CRF. Because CRF is believed to be an important neurotransmitter that activates and coordinates the endocrine, behavioral and automatic responses to stress, CRF receptor antagonists of the present invention may be useful in the treatment of neuropsychiatric disorders. Neuropsychiatric disorders which may be treatable by the CRF receptor antagonists of this invention include affective disorders such as depression; anxiety-related disorders such as generalized anxiety disorder, panic disorder, obsessive-compulsive disorder, abnormal aggression, cardiovascular abnormalities such as unstable angina and reactive hypertension; and feeding disorders such as anorexia nervosa, bulimia, and irritable bowel syndrome. CRF antagonists may also be useful in treating stress-induced immune suppression associated with various diseases states, as well as stroke. Other uses of the CRF antagonists of this invention may include treatment of inflammatory conditions (such as rheumatoid arthritis, uveitis, asthma, inflammatory bowel disease and G.I. motility), pain, Cushing's disease, infantile spasms, epilepsy and other seizures in both infants and adults, and various substance abuse and withdrawal (including alcoholism).

In another embodiment of the invention, pharmaceutical compositions containing one or more CRF receptor antagonists are disclosed. For the purposes of administration, the compounds of the present invention may be formulated as pharmaceutical compositions. Pharmaceutical compositions of the present invention comprise a CRF receptor antagonist of the present invention (i.e., a compound of structure (I)) and a pharmaceutically acceptable carrier and/or diluent. The CRF receptor antagonist is present in the composition in an amount which is effective to treat a particular disorder—that is, in an amount sufficient to achieve CRF receptor antagonist activity, and preferably with acceptable toxicity to the patient. The pharmaceutical compositions of the present invention may include a CRF receptor antagonist in an amount from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a CRF receptor antagonist, diluents, dispersing and surface active agents, binders, and lubricants. One skilled in this art may further formulate the CRF receptor antagonist in an appropriate manner, and in accordance with accepted practices, such as those disclosed in Remington's Pharmaceutical Sciences, Gennaro, Ed., Mack Publishing Co., Easton, Pa. 1990.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound.

With regard to stereoisomers, the compounds of structure (I) may have chiral centers and may occur as racemates, racemic mixtures and as individual enantiomers or diastereomers. All such isomeric forms are included within the present invention, including mixtures thereof. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

In another embodiment, the present invention provides a method for treating a variety of disorders or illnesses, including endocrine, psychiatric and neurological disorders or illnesses. Such methods include administering of a compound of the present invention to a mammal (e.g., a person) in an amount sufficient to treat the disorder or illness. Such methods include systemic administration of a CRF receptor antagonist of this invention, preferably in the form of a pharmaceutical composition. As used herein, systemic administration includes oral and parenteral methods of administration. For oral administration, suitable pharmaceutical compositions of CRF receptor antagonists include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions which may contain, in addition to the CRF receptor antagonist, buffers, antioxidants, bacteriostats, and other additives commonly employed in such solutions.

In another embodiment, the present invention permits the diagnostic visualization of specific sites within the body by the use of radioactive or non-radioactive pharmaceutical agents Use of a compound of the present invention may provide a physiological, functional, or biological assessment of a patient or provide disease or pathology detection and assessment. Radioactive pharmaceuticals are employed in scintigraphy, positron emission tomography (PET), computerized tomography (CT), and single photon emission computerized tomography (SPECT.) For such applications, radioisotopes are incorporated of such elements as iodine (I) including ¹²³I (PET), ¹²⁵I (SPECT), and ¹³¹I, technetium (Tc) including ⁹⁹Tc (PET), phosphorus (P) including ³¹P and ³²P, chromium (Cr) including ⁵¹Cr, carbon (C) including ¹¹C, fluorine (F) including ¹⁸F, thallium (Tl) including ²⁰¹Tl, and like emitters of positron and ionizing radiation. Non-radioactive pharmaceuticals are employed in magnetic resonance imaging (MRI), fluoroscopy, and ultrasound. For such applications, isotopes are incorporated of such elements as gadolinium (Gd) including ¹⁵³Gd, iron (Fe), barium (Ba), manganese (Mn), and thallium (Tl). Such entities are also useful for identifying the presence of particular target sites in a mixture and for labeling molecules in a mixture.

As mentioned above, administration of a compound of the present invention can be used to treat a wide variety of disorders or illnesses. In particular, the compounds of the present invention may be administered to a mammal for the treatment of depression, anxiety disorder, panic disorder, obsessive-compulsive disorder, abnormal aggression, unstable angina, reactive hypertension, anorexia nervosa, bulimia, irritable bowel syndrome, stress-induced immune suppression, stroke, inflammation, pain, Cushing's disease, infantile spasms, epilepsy, and substance abuse or withdrawal.

The following examples are provided for purposes of illustration, not limitation.

### EXAMPLES

Preparative HPLC-MS

Platform: Shimadzu HPLC equipped with a Gilson 215 auto-sampler/fraction collector, UV detector and a PE Sciex API150EX mass detector;

HPLC column: BHK ODS-O/B, 5μ, 30×75 mm

HPLC gradient: 35 mL/minute, 10% acetonitrile in water to 100% acetonitrile in 7 minutes, maintaining 100% acetonitrile for 3 minutes, with 0.025% TFA.

Analytical HPLC-MS—Method 1

Platform: Agilent 1100 series: equipped with auto-sampler, UV detector (220 nM and 254 nM) and MS detector (APCI);

HPLC column: Phenomenex Synergi-Max RP, 2.0×50 mm column;

HPLC gradient: 1.0 mL/minute, from 5% acetonitrile in water to 95% acetonitrile in water in 13.5 min, maintaining 95% acetonitrile for 2 min, both acetonitrile and water having 0.025% TFA.

Analytical HPLC-MS—Method 2

Platform: Agilent 1100 series: equipped with auto-sampler, UV detector (220 nM and 254 nM), MS detector (APCI) and Berger FCM 1200 CO₂ pump module;

HPLC column: Berger Pyridine, PYR 60A, 6μ, 4.6×150 mm column;

HPLC gradient: 4.0 mL/minute, 120 bar; from 10% methanol in supercritical CO₂ to 60% methanol in supercritical CO₂ in 1.67 minutes, maintaining 60% for 1 minute. Methanol has 1.5% water. Backpressure regulated at 140 bar.

Abbreviations:

AA: Acetic anhydride

Boc-Phe-CHO: (S)-(tertbutoxycarbonylamino)-3-phenylpropional

BOC: tert-butoxycarbonyl

DCM: dichloromethane

DMF: dimethylformamide

DMSO: dimethylsulfoxide

EDC: 1-(3-dimethylaminopropyl)-3-ethylcarbodiimide hydrochloride

FMOC: N-(9-fluorenylmethoxycarbonyl)

HOBt: 1-hydroxybenzotriazole hydrate

NaBH(OAc)₃: Sodium Triacetoxyborohydride

Pd—C: Palladium (10%) on Carbon

TFA: Trifluoroacetic acid

THF: Tetrahydrofuran

The CRF receptor antagonists of this invention may be prepared by the methods disclosed in Examples 1 to 6. Example 7 presents a method for determining the receptor binding activity (Kᵢ).

### Example 1

### Synthesis of Reagent 5-Chloro-1-(1-ethyl-propyl)-7-methyl-1,2,3,4-tetrahydro-pyrido[3,4-b]pyrazine

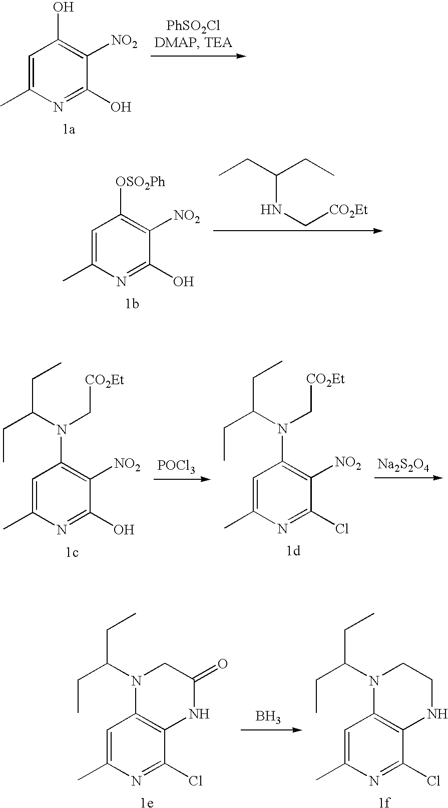 Step 1A:

To a suspension of 3-nitro-6-methyl-pyridine-2,4-diol (1a, 25.5 g) and DMAP (0.92 g) in THF (250 mL) was added TEA (16.7 g) dropwise. The resulting suspension was heated to reflux while benzenesulfonyl chloride (29.2 g) was added dropwise and the mixture was refluxed for an additional 1 hr following completion of the addition. Evaporation of solvent yielded crude 1b.

Step 1B:

The residue from Step 1A (entire amount) was suspended in MeCN (250 mL.) DMAP (1.0 g) was added followed by dropwise addition of a solution of the amino ester (26.0 g.) in MeCN. The mixture was heated to reflux overnight. After evaporation of solvent, the residue was extracted between EtOAc and aqueous NaHCO₃, then the organic layer was dried over sodium sulfate, filtered, and concentrated to yield 1c.

Step 1C:

The crude 1c was dissolved in MeCN (50 mL) and refluxed with POCl₃ (2.0 eq) overnight. The mixture was poured onto ice, then neutralized with sodium carbonate. The mixture was extracted with ethyl acetate. The combined organic extracts were dried over sodium sulfate, filtered, and concentrated. The residue was purified by silica gel chromatography to afford 1d (10.47 g).

Step 1D:

To a solution of Na₂S₂O₄ (26.56 g) and NaHCO₃ (12.82 g) in water (100 mL) was added a solution of 1d (10.45 g) in MeCN (80 mL) dropwise at room temperature. After stirring for 2 hr, the MeCN was evaporated and the residue was extracted with EtOAc. The combined organic extracts were dried over sodium sulfate, filtered, and concentrated to provide crude compound 1e (6.20 g).

Step 1E:

To crude compound 1e (6.20 g) in dry THF (20 mL) was added borane (1M solution in THF, 3.0 eq) slowly. After stirring at room temperature overnight, methanol was carefully added, then the solvent was evaporated to provide compound 1f (3.1 g).

### Example 2

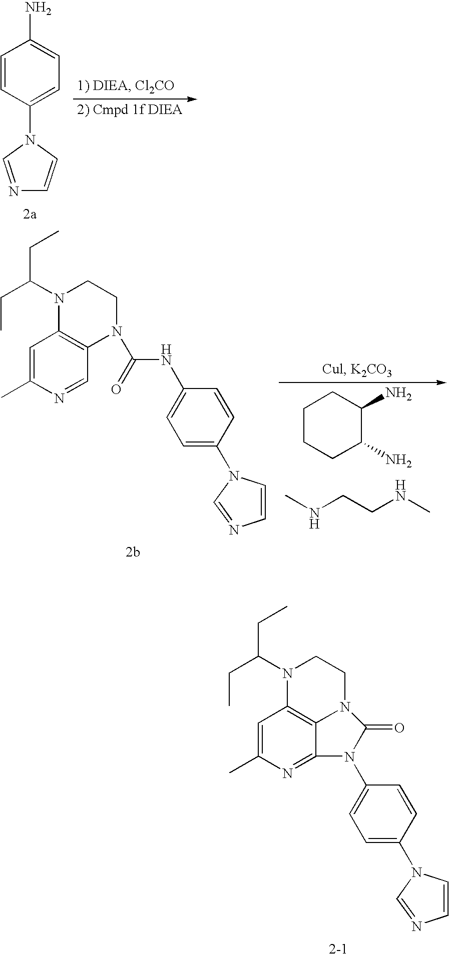 Step 2A:

To a solution of aniline 2a (23 mg) and DIEA (26 mg) in dry DCM (1.0 mL) was added phosgene (250 uL, 20% in toluene) very slowly at room temperature. The resulting mixture was stirred overnight and evaporated to dryness. To the residue was added a solution of compound 1f (Example 1, 25 mg) and DIEA (480 mg) in dry DCM. The resulting mixture was stirred at room temperature 48 hr prior to quenching with water. The organic layer was dried over MgSO₄ and evaporated to dryness.

Step 2B:

The residue from Step 2A (entire amount) was dissolved in 1,4-dioxane (1.0 mL) and stirred vigorously prior to the sequential addition of Cul (20 mg,) K₂CO₃ (40 mg,) trans-1,2-diaminocyclohexane (12 uL,) and N,N′-dimethylethylenediamine (12 uL.) The resulting slurry was heated in a sealed tube at 110° C. overnight which gave after purification via preparative LC-MS compound 2-1 (30.8 mg.) as a TFA salt.

By employing 3-amino-6-(morpholino-4-yl)pyridine in Step 2A, Cpd 2-2 was synthesized as shown in the table:

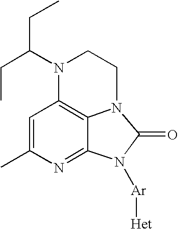

| | | | | | | HPLC |
|---|---|---|---|---|---|---|
| | Ar | Het | MW | [MH]⁺ | t_{R} | Meth. |
| 2-1 | 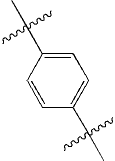 | 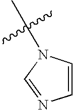 | 402.499 | 403 | 1.143 | 2 |
| 2-2 | 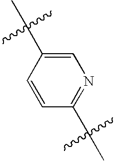 | 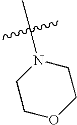 | 422.530 | 423.2 | 4.041 | 1 |

### Example 3

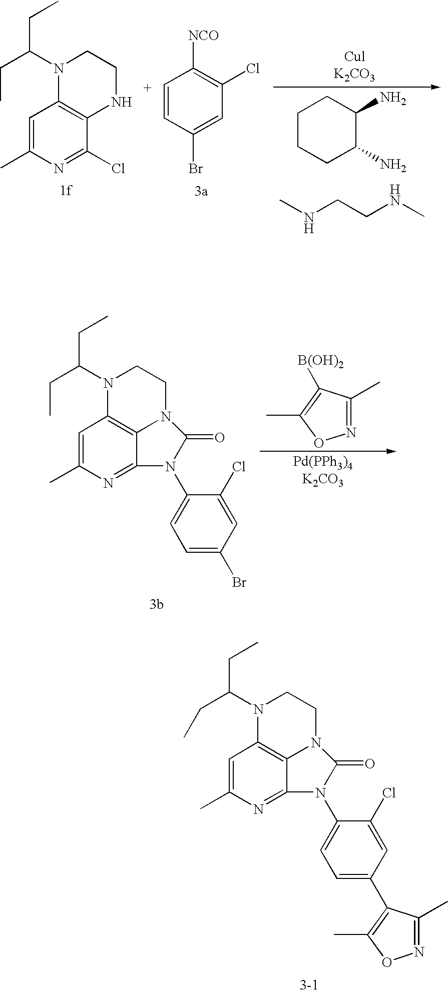 Step 3A:

A mixture of compound 1f (253 mg, Example 1) and 2-chloro-4-bromophenyl isocyanate (232 mg) in 1,4-dioxane (2.5 mL) was stirred at room temperature for 5 hr. To the resulting mixture was added Cul (100 mg,) K₂CO₃ (414 mg,) trans-1,2-diaminocyclohexane (50 uL,) and N,N′-dimethylethylenediamine (50 uL) in turn prior to heating overnight in a sealed tube at 110° C. The resulting compound 3b (190 mg) was purified by silica gel chromatography.

Step 3B:

A mixture of compound 3b (25 mg) together with (3,5-dimethyl-isoxazole)-4-boronic acid (0.12 mmol), tetrakis(triphenylphosphine)palladium(0) (0.01 mmol), and K₂CO₃ (0.25 mmol) was heated (100° C.) in dioxane/water overnight in a sealed tube. Compound 3-1 (8.4 mg) was obtained as a TFA salt after purification via preparative LC-MS. By varying the structure of the heterocycle boronic acid, the compounds in the following table were synthesized:

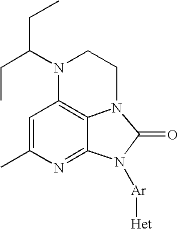

| | Ar | Het | MW | [MH]⁺ | t_{R}* |
|---|---|---|---|---|---|
| 3-1 | 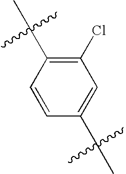 | 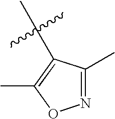 | 465.982 | 610.3 | 1.009 |
| 3-2 | 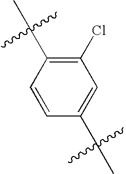 | 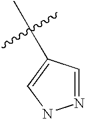 | 436.944 | 437 | 1.422 |
| 3-3 | 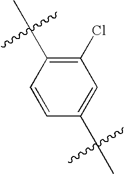 | 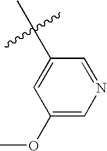 | 477.993 | 478 | 1.162 |
| 3-4 | 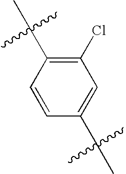 | 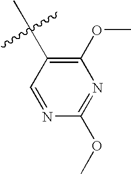 | 509.01 | 509 | 1.098 |

*All t_{R} reported for Analytical HPLC Method 2.

### Example 4

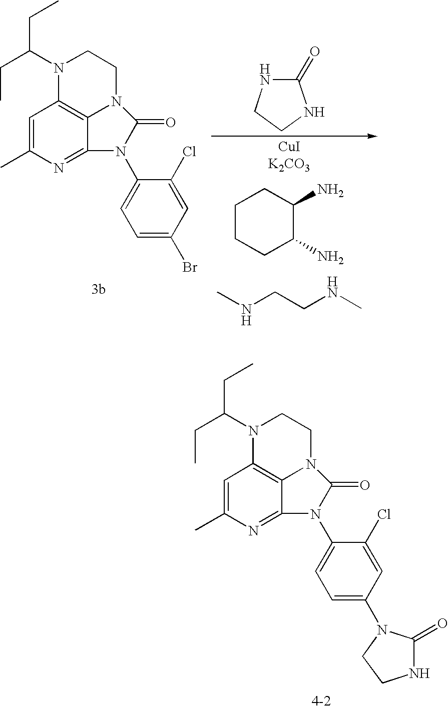 Step 4A:

Compound 3b (25 mg) was mixed with imidazolidin-2-one and subjected to the general procedure of Cul-mediate coupling described in Example 3. Compound 4-2 (3.0 mg) was obtained after purification via preparative LC-MS. By varying the heterocycle reactant the following compounds were synthesized.

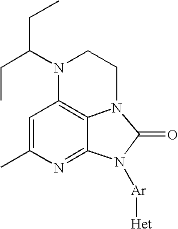

| | Ar | Het | MW | [MH]⁺ | t_{R}* |
|---|---|---|---|---|---|
| 4-1 | 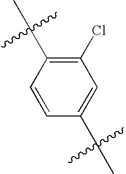 | 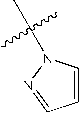 | 436.944 | 437 | 1.168 |
| 4-2 | 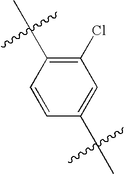 | 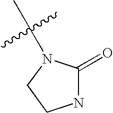 | 454.959 | 455 | 1.419 |
| 4-3 | 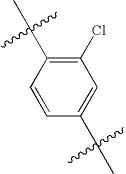 | 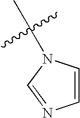 | 436.944 | 437 | 1.245 |

*All t_{R} reported for Analytical HPLC Method 2.

### Example 5

### Synthesis of Reagent 3-Chloro-4-[5-(1-ethyl-propyl)-7-methyl-2-oxo-4,5-dihydro-3H-1,2a,5,8-tetraaza-acenaphthylen-1-yl]-benzonitrile

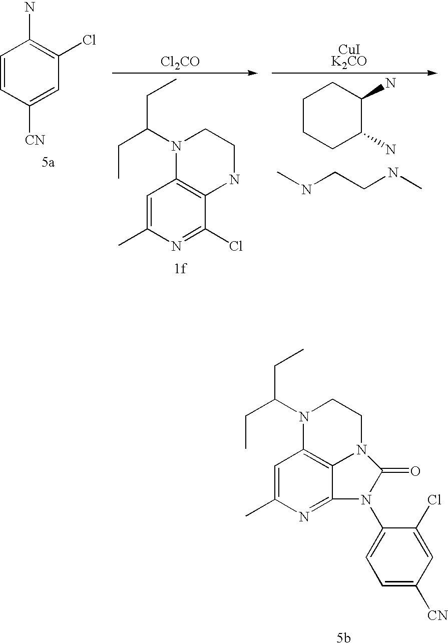 Step 5A:

To a solution of phosgene (2.3 mL, 20% in toluene) in dry DCM (20 mL) was added a solution of 2-chloro-4-cyanoaniline (367 mg) and DIEA (372 mg) in dry DCM slowly. The resulting mixture was stirred at room temperature for 1 hr prior to evaporation to dryness. The residue was dissolved in DCM (10 mL) to which a solution of compound 1f (Example 1, 510 mg) and DIEA (310 mg) in DCM was added. The resulting mixture was stirred overnight at room temperature. Aqueous sodium bicarbonate was added and the mixture was extracted with DCM. The organic phase was dried over MgSO₄ and evaporated to dryness.

Step 5B:

The residue of Step 5A was subjected to the general Cul-mediated coupling reaction conditions described in Example 3 to give cyano compound 5b (313 mg) after chromatographic purification.

### Example 6

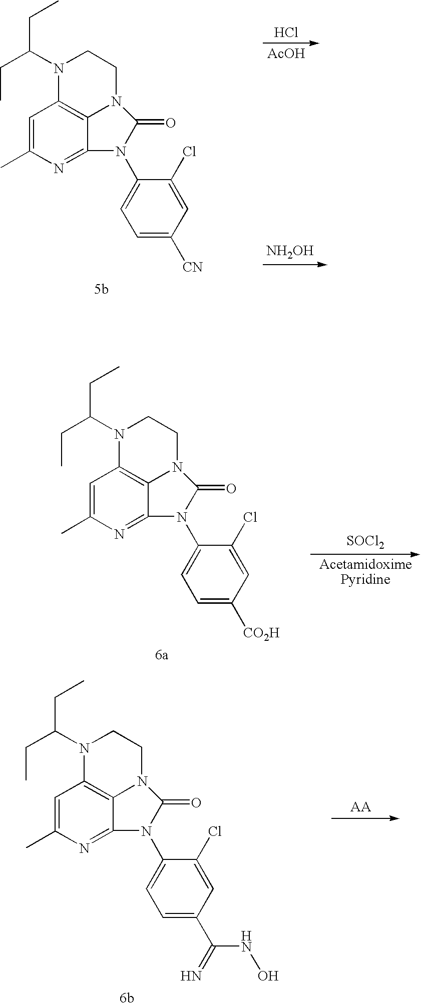 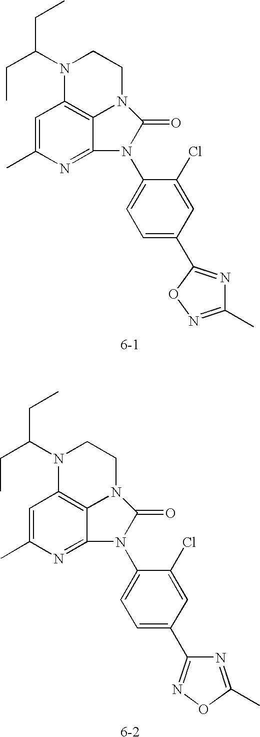 Step 6A (Upper Branch):

Compound 5b (150 mg) was heated in a solution with HCl (aq.) in AcOH at 80° C. for 8 hr. After evaporation to dryness, the crude acid 6a was heated with SOCl₂ (100 uL) in chloroform at 60° C. for 2 hr. After evaporation to dryness, the residue was mixed with acetamidoxime and pyridine (0.8 mL), and the resulting mixture was heated at 110° C. for 24 hr. The resulting compound 6-1 (4.0 mg) was obtained as a TFA salt following preparative LC-MS purification.

Step 6B (Lower Branch):

To a suspension of hydroxylamine hydrochloride (8.5 mg) in ethanol was added NaOMe (25% wt in MeOH, 30 uL) at room temperature with stirring. Compound 5b (40 mg) was added and the resulting mixture was heated at 80° C. for 4 hr. After aqueous workup, the resulting amidoxime was heated with acetic anhydride (0.2 mL) in pyridine (0.8 mL) at 110° C. for 24 hr. The resulting compound 6-2 (3.7 mg) was obtained as a TFA salt following preparative LC-MS purification.

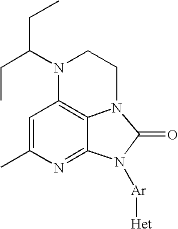

| | Ar | Het | MW | [MH]⁺ | t_{R}* |
|---|---|---|---|---|---|
| 6-1 | 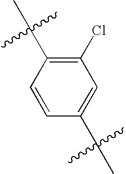 | 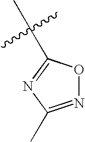 | 452.944 | 453 | 0.961 |
| 6-2 | 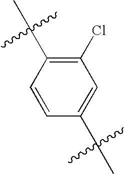 | 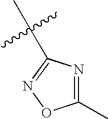 | 452.944 | 453 | 1.045 |

*All t_{R} reported for Analytical HPLC Method 2.

### Example 7

### CRF Receptor Binding Activity

The compounds of this invention may be evaluated for binding activity to the CRF receptor by a standard radioligand binding assay as generally described by Grigoriadis et al. (Mol. Pharmacol vol 50, pp 679-686, 1996) and Hoare et al. (Mol. Pharmacol vol 63 pp 751-765, 2003.) By utilizing radiolabeled CRF ligands, the assay may be used to evaluate the binding activity of the compounds of the present invention with any CRF receptor subtype.

Briefly, the binding assay involves the displacement of a radiolabeled CRF ligand from the CRF receptor. More specifically, the binding assay is performed in 96-well assay plates using 1-10 μg cell membranes from cells stably transfected with human CRF receptors. Each well receives about 0.05 ml assay buffer (e.g., Dulbecco's phosphate buffered saline, 10 mM magnesium chloride, 2 mM EGTA) containing compound of interest or a reference ligand (for example, sauvagine, urocortin I or CRF), 0.05 ml of [¹²⁵I] tyrosine-sauvagine (final concentration ˜150 pM or approximately the K_{D} as determined by Scatchard analysis) and 0.1 ml of a cell membrane suspension containing the CRF receptor. The mixture is incubated for 2 hours at 22° C. followed by separation of the bound and free radioligand by rapid filtration over glass fiber filters. Following three washes, the filters are dried and radioactivity (Auger electrons from ¹²⁵I) is counted using a scintillation counter. All radioligand binding data may be analyzed using the non-linear least-squares curve-fitting programs Prism (GraphPad Software Inc) or XLfit (ID Business Solutions Ltd).

### Example 8

### CRF-Stimulated Adenylate Cyclase Activity

The compounds of the present invention may also be evaluated by various functional testing. For example, the compounds of the present invention may be screened for CRF-stimulated adenylate cyclase activity. An assay for the determination of CRF-stimulated adenylate cyclase activity may be performed as generally described by Battaglia et al. (Synapse 1:572, 1987) with modifications to adapt the assay to whole cell preparations.

More specifically, the standard assay mixture may contain the following in a final volume of 0.1 ml: 2 mM L-glutamine, 20 mM HEPES, and 1 mM IMBX in DMEM buffer. In stimulation studies, whole cells with the transfected CRF receptors are plated in 96-well plates and incubated for 30 min at 37° C. with various concentrations of CRF-related and unrelated peptides in order to establish the pharmacological rank-order profile of the particular receptor subtype. Following the incubation, cAMP in the samples is measured using standard commercially available kits, such as cAMP-Screen™ from Applied Biosystems. For the functional assessment of the compounds, cells and a single concentration of CRF or related peptides causing 50% stimulation of cAMP production are incubated along with various concentrations of competing compounds for 30 min at 37° C., and cAMP determined as described above.

1. A compound having the following structure: 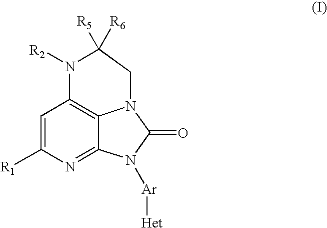
or a pharmaceutically acceptable salt, ester, solvate, stereoisomer or prodrug thereof,
wherein:
R₁ and R₂ are the same or different and independently are hydrogen, alkyl, or substituted alkyl;
Ar is phenyl, phenyl substituted by 1 or 2 R₃, pyridyl or pyridyl substituted by 1 or 2 R₃;
R₃ at each occurrence is independently alkyl, substituted alkyl, alkoxy, substituted alkoxy, cyano, halogen, alkylsulfinyl, or alkylsulfonyl;
Het is heterocycle or heterocycle substituted with 1 or 2 R₄;
R₄ at each occurrence is independently alkyl, substituted alkyl, alkoxy, substituted alkoxy, halogen, cyano or oxo; and
R₅ and R₆ are the same or different and independently are hydrogen, alkyl or substituted alkyl.

2. A compound according to claim 1, wherein R₁ is alkyl.

3. A compound according to claim 1, wherein R₂ is alkyl or substituted alkyl.

4. A compound according to claim 1, wherein Ar is phenyl substituted by 1 R₃.

5. A compound according to claim 4, wherein R₃ is alkyl, substituted alkyl, alkoxy, cyano or halogen.

6. A compound according to claim 4, wherein R₃ is halogen.

7. A compound according to claim 1, wherein Het is heterocycle substituted with 1 R₄.

8. A compound according to claim 7, wherein R₄ is alkyl, substituted alkyl, or alkoxy.

9. A compound according to claim 7, wherein R₄ is oxo.

10. A compound according to claim 3, wherein R₅ at each occurrence is hydrogen.

11. A compound according to claim 10, wherein Ar is phenyl, pyridyl or phenyl substituted by one R₃, wherein R₃ is halogen.

12. A compound according to claim 11, wherein Ar is phenyl substituted by one R₃, and wherein R₃ is chloro.

13. A compound according to claim 12, wherein Het is heterocycle.

14. A compound according to claim 13, wherein Het is pyrazole.

15. A compound according to claim 12, wherein Het is heterocycle substituted by one or two R₄.

16. A compound according to claim 15, wherein Het is dimethylisoxazole.

17. A composition comprising a compound according to claim 1 and a pharmaceutically acceptable carrier or diluent.

18. A method for treating a disorder manifesting hypersecretion of CRF in a mammal comprising administering to the mammal a pharmaceutically effective amount of the pharmaceutical composition of claim 17.

19. A method according to claim 18, wherein the disorder is an affective disorder, an axiety-related disorder, a feeding disorder, a stress-induced immunosuppressive disorder, an inflammatory disorder or a substance abuse or withdrawal disorder.

20. A method according to claim 18, wherein the disorder is irritable bowel syndrome.

21. A method according to claim 18, wherein the disorder is depression.

22. A method according to claim 18, wherein the disorder is anxiety.

23. A method according to claim 18, wherein the disorder is obsessive-compulsive disorder.

24. A method according to claim 18, wherein the disorder is stroke.
